(19) United States
(12) Patent Application Publication
(10) Pub. No.: US 2025/0136554 A1
(43) Pub. Date: May 1, 2025

(54) CRYSTALLINE FORM OF ANSERINE, AND METHOD OF PRODUCING THE CRYSTALLINE FORM OF ANSERINE

(71) Applicant: TOKAI BUSSAN CO., LTD., Tokyo (JP)

(72) Inventors: Akira YONEYAMA, Tokyo (JP); Hiroki NAKANISHI, Tokyo (JP); Chikako TAKAHASHI, Tokyo (JP); Chiaki SANO, Tokyo (JP); Yoshio KAWAI, Tokyo (JP); Yohsuke KOYAMA, Tokyo (JP)

(73) Assignee: TOKAI BUSSAN CO., LTD., Tokyo (JP)

(21) Appl. No.: 18/694,266

(22) PCT Filed: Sep. 22, 2022
(86) PCT No.: PCT/JP2022/035342

§ 371 Date: Mar. 21, 2024

(30) Foreign Application Priority Data
Sep. 24, 2021 (JP) 2021-155549

(51) Int. Cl. C07D 233/64

(52) U.S. Cl. CPC C07D 233/64

(57) ABSTRACT

The objective of the present invention is to provide to a highly pure crystalline form of anserine and a method of producing the crystalline form. The above objective is solved by a crystalline form of anserine, characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation); a crystalline form of anserine, characterized by a melting point of 240±2° C. by differential thermal analysis and the like.

### TECHNICAL FIELD

The present invention relates to a novel crystalline form of anserine and a method of producing the crystalline form of anserine.

### BACKGROUND ART

Imidazole dipeptides are a dipeptide in which histidine or a histidine derivative having an imidazole group is bound to an amino acid. One of the imidazole dipeptides is anserine (L-anserine; β-alanyl-1-methylhistidine; CAS No.: 584-85-0). Anserine is a dipeptide composed of L-methylhistidine and β-alanine, and is abundant in muscles of marine organisms such as salmon, tuna, bonito and shark, and of birds.

Anserine is a palatable ingredient, and further has an attracted attention as a functional ingredient having physiological effects such as anti-fatigue, antioxidant, hypotensive, anti-inflammatory and uric acid lowering effects.

The present inventors have been able to produce a highly pure anserine from chicken meat (see, for example, Patent Document 1).

Another imidazole dipeptide known as with anserine is carnosine. Known methods of obtaining a crystalline form of carnosine includes a method including precipitating a L-carnosine crystal by evaporating water from an aqueous solution dissolving a crude L-carnosine to obtain a slurry solution of L-carnosine, and then heating the slurry solution and leaving for a certain period of time followed by gradually adding alcohol and mixing the mixture to obtain a highly pure crystalline form of L-carnosine (see, for example, Patent Document 2).

Another known method of obtaining a crystalline form of carnosine includes cooling an aqueous solution dissolving L-carnosine and a neutral salt, or dropping or adding an aqueous solution dissolving a base to an aqueous solution dissolving L-carnosine and an acid (see, for example, Patent Document 3).

Besides, a method including chemically synthesizing anserine using phthalyl-L-carnosine as a starting material is known (see, for example, Patent Document 4).

### CITATION LIST

### Patent Document

[Patent Document 1] JP 6769643 B
[Patent Document 2] JP 5448588 B
[Patent Document 3] JP 2018-203690 A
[Patent Document 4] JP 4463515 B

### SUMMARY OF THE INVENTION

### Problems to be Solved by the Invention

In general, an animal extract includes two or more imidazole dipeptides, according to the type and part of the animal. Therefore, even if a highly pure anserine-containing composition is obtained by the method described in Patent Document 1, the composition may contain a certain amount of other imidazole dipeptides such as carnosine, and may also contain contaminants such as creatinine. As a result, the method described in Patent Document 1 does not produce a highly pure crystalline form of anserine, e.g., with a purity of 99% or higher.

The present inventors found out that the solubility of anserine in water or organic solvent is different from that of carnosine. Carnosine has less solubility in water or hydrous organic solvent. Therefore, the use of the method described in Patent Document 2 or 3 results in a highly pure crystalline form of carnosine by concentrating or salting out an aqueous solution of carnosine to obtain a crystalline form of carnosine, and then gradually adding organic solvent to the solution containing the crystalline form of carnosine to dissolve impurities but to precipitate the crystalline form of carnosine.

However, anserine has greater solubility in water or hydrous organic solvent. Therefore, the method described in Patent Document 2 or 3 cannot produce not only a highly pure crystalline form of anserine but also any crystalline form of anserine.

On the other hand, anserine can be obtained by the method described in Patent Document 4. However, the anserine product obtained by the method described in Patent Document 4 has a less purity of 99.7% and further contains 0.3% carnosine. There have been very little known a crystalline form of anserine with a high purity of 99.8% or more and a method of producing the crystalline form of anserine.

Accordingly, an object of the present invention is to provide a highly pure crystalline form of anserine and a method of producing the crystalline form.

### Means for Solving the Problem

During earnestly studying to solve the above problems, the present inventors found out that concentrating an aqueous solution containing anserine, as in the method described in Patent Document 2, could not produce a crystalline form of anserine but instead produced a candy-like viscous solution. In addition, when a crude anserine obtained from an animal extract with a pH near neutral was employed, a large amount of chloride ions remained, thereby hindering the crystallization of anserine.

The present inventors further repeated trial and error to try to obtain a highly pure crystalline form of anserine. As a result, the present inventors achieved to efficiently separate and remove chloride ions with the use of ion exchange treatment using an OH-type strongly basic anion exchange resin in the case of using a crude anserine derived from an animal extract with a pH near neutral. In this case, the present inventors also found out that a crystalline form of anserine could be obtained by concentrating the resulting ion-exchange treatment solution to obtain a candy-like viscous solution, and instantaneously adding organic solvent to the candy-like viscous solution such that the final concentration of organic solvent became at a predetermined level, and mixing the mixture. However, the resulting crystalline form of anserine had a purity less than 99% and contained 1% or more creatinine.

The present inventors then isolated the crystalline form of anserine as a cake, and further washed the resulting cake using the high concertation of organic solvent in a stepwise manner. As a result, the present inventors surprisingly succeeded in obtaining a crystalline form of anserine with a purity of 99% or more. As such, the present invention has been completed on the basis of the findings and successful examples that were found or obtained by the present inventors.

According to the present invention, there are provided the following embodiments:
[I-1] A crystalline form of anserine, characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation).
[I-2] A crystalline form of anserine, characterized by a melting point of 240±2° C. by differential thermal analysis.
[I-3] The crystalline form of anserine according to any one of [I-1] to [I-2], wherein the purity of anserine is equal to or more than 99.8% and/or the purity of carnosine is less than 0.2%.
[I-4] A food composition, pharmaceutical composition or cosmetic composition, comprising the crystalline form according to any one of [I-1] to [I-3].
[I-5] A method of producing a crystalline form of anserine, comprising instantaneously adding to an aqueous solution comprising a crude anserine with a purity equal to or more than 70% organic solvent at such an amount that the final concentration becomes equal to or more than 70% to obtain a slurry;
   subjecting the slurry to solid-liquid separation treatment to obtain a cake; and
   subjecting the cake to washing treatment using 85% or more hydrous organic solvent to obtain the crystalline form of anserine.
[I-6] The method according to [I-5], wherein the aqueous solution comprising a crude anserine is an aqueous solution comprising a crude anserine obtained from an animal extract.
[I-7] The method according to any one of [I-5] to [I-6], wherein the aqueous solution comprising a crude anserine is an aqueous solution comprising a crude anserine subjected to ion exchange treatment using an OH type strongly basic anion exchange resin.
[I-8] The method according to any one of [I-5] to [I-6], wherein the aqueous solution comprising a crude anserine is an aqueous solution comprising a crude anserine subjected to ion exchange treatment using an OH type strongly basic anion exchange resin and concentration treatment.
[I-9] The method according to [I-8], wherein the concentration treatment is decompression concentration treatment to be carried out until Brix of the aqueous solution becomes in the range from 50% to 70%.
[I-10] The method according to any one of [I-5] to [I-9], wherein the washing treatment is washing treatment in which washing using 85% to 94% hydrous organic solvent and washing using 95% to 99.9% hydrous organic solvent are sequentially carried out.
[I-11] The method according to any one of [I-5] to [I-10], wherein the organic solvent used is each independently at least one organic solvent selected from the group consisting of methanol, ethanol, 2-propanol and acetone.
[I-12] The method according to any one of [I-6] to [I-11], wherein the animal extract is an animal extract derived from at least one animal selected from the group consisting of bonito, tuna, salmon, eel, shark, cattle and chicken.
[II-1] A crystalline form of anserine, characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation), a melting point of 240±2° C. by differential thermal analysis, the purity of anserine equal to or more than 99.8%, and the purity of carnosine less than 0.2%.
[II-2] A food composition, pharmaceutical composition or cosmetic composition, comprising the crystalline form according to [II-1].
[II-3] A method of producing a crystalline form of anserine, comprising instantaneously adding to an aqueous solution comprising a crude anserine with a purity equal to or more than 70% an ethanol solution at such an amount that the final concentration is equal to or more than 70% and agitating the mixture to obtain a slurry;
   subjecting the slurry to solid-liquid separation treatment to obtain a cake; and
   subjecting the cake to washing treatment using a 85% to 94% hydrous ethanol solution and washing treatment using a 95% to 99.9% hydrous ethanol solution in a stepwise manner to obtain the crystalline form of anserine according to [II-1].
[II-4] The method according to [II-3], wherein the aqueous solution comprising a crude anserine is an aqueous solution comprising a crude anserine obtained from an animal extract.
[II-5] The method according to any one of [II-3] to [II-4], wherein the aqueous solution comprising a crude anserine is an aqueous solution comprising a crude anserine subjected to ion exchange treatment using an OH type strongly basic anion exchange resin.
[II-6] The method according to any one of [II-3] to [II-4], wherein the aqueous solution comprising a crude anserine is an aqueous solution comprising a crude anserine subjected to ion exchange treatment using an OH type strongly basic anion exchange resin and concentration treatment.
[II-7] The method according to [II-6], wherein the concentration treatment is decompression concentration treatment to be carried out until Brix of the aqueous solution is in the range from 50% to 70%.
[II-8] The method according to any one of [II-4] to [II-7], wherein the animal extract is an animal extract derived from at least one animal selected from the group consisting of bonito, tuna, salmon, eel, shark, cattle and chicken.

### Effect of the Invention

According to the present invention, the crystalline form of anserine with a purity of 99% or more can be obtained. Furthermore, the method according to one embodiment of the present invention is a simple and economical method, so that the method can produce a highly pure crystalline form of anserine on an industrial scale. According to the present invention, it is also possible to obtain a crystalline form of anserine with possibly little carnosine and creatinine, even if an animal extract, which is abundant in carnosine and creatinine, is used as an anserine source.

### BRIEF DESCRIPTION OF THE DRAWINGS

FIG. 1 is the graph illustrating the X-ray powder diffraction pattern of Anserine crystal III, as shown in Examples described below.

FIG. 2A is the graph comparing the X-ray powder diffraction pattern of Anserine crystal III with the X-ray powder diffraction pattern of L-anserine nitrate, as shown in Examples described below.

FIG. 2B is the graph comparing the X-ray powder diffraction pattern of Anserine crystal III with the X-ray powder diffraction pattern of L-carnosine, as shown in Examples described below.

FIG. 3 is the graph showing the data of differential thermal analysis of Anserine crystal III, as shown in Examples described below.

FIG. 4 is the polarized light micrograph of Anserine crystal II, as shown in Examples described below.

FIG. 5 is the graph showing the measurement results of the solubility of each crystalline form of anserine, carnosine and creatinine in various concentrations of ethanol, as shown in Examples described below.

FIG. 6 is the graph showing the measurement results of the solubility of each crystalline form of anserine, carnosine and creatinine in various types of organic solvent, as shown in Examples described below.

FIG. 7 is the graph evaluating the influence of pH on the solubility of crystalline form of anserine in hydrous ethanol solution, as shown in Examples described below.

### DESCRIPTION OF EMBODIMENTS

While each embodiment of the present invention will now be described in detail, the present invention is not limited only by the matters of this section, and may take various forms to the extent that its objective can be achieved.

Unless otherwise specified, each term in this specification is used in the sense normally used by those skilled in the art and should not be construed as having an unduly restrictive meaning. Also, any speculations and theories herein are made on the basis of the knowledge and experiences of the present inventors and as such, the present invention is not bound by any such speculations and theories.

The term “RV” means a multiple number of flow rate of solvent relative to an amount of resin. For example, if the two times amount of animal extract relative to the amount of resin is passed through the resin, RV makes 2.

The term “SV” is Space Velocity, which means a ratio per hour of a liquid amount (volume) flowed to a resin amount (volume). For example, if 5 m³ of liquid is passed through 1 m³ of resin for an hour, SV makes 5.

The term “purity” is synonymous with content and concentration and, unless otherwise mentioned, is calculated based on the ratio of the dry mass of the component relative to the dry mass of solids. The amount of anserine is determined by the HPLC method described in Examples below.

The wording “to” for indicating a range of values is intended to include values preceding and following the wording; for example, “0% to 100%” means a range from 0% or more and 100% or less. The terms “more than” and “less than” mean the lower and upper limits without including a value following the term, respectively. For example, “more than 1” means a value beyond 1, and “less than 100” means a value below 100.

The term “and/or” means either any one of, any combination of two or more of, or combination of all of listed related items.

The terms “include,” “comprise,” and “contain” mean that an element(s) other than an element(s) as explicitly indicated can be added as inclusions, which are, for example, synonymous with “at least include,” but encompasses the meaning of “consist of” and “substantially consist of”. In other words, the terms may mean, for example, to include an element(s) as explicitly indicated as well as any one element or any two or more elements, to consist of an element(s) as explicitly indicated, or to substantially consist of an element(s) as explicitly indicated. Such elements include limitations such as components, steps, conditions, and parameters.

The number of digits of an integer equals to its significant figure. For example, 1 has one significant figure and 10 has two significant figures. For a decimal number, the number of digits after a decimal point equals to its significant figure. For example, 0.1 has one significant figure and 0.10 has two significant figures.

### [Crystalline Form According to One Embodiment of the Present Invention]

One embodiment of the present invention is a crystalline form of anserine. The crystalline form of anserine according to one embodiment of the present invention is a crystalline form of anserine in the free-form. The crystalline form according to one embodiment of the present invention is characterized by diffraction angles (2θ) of diffraction peaks in an X-ray powder diffraction pattern and/or a melting point by differential thermal analysis.

One aspect of the present invention is a crystalline form of anserine characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation).

The value of diffraction angle 2θ of diffraction peak in the X-ray powder diffraction pattern is construed to be the above-mentioned value±0.2°, since an error can occur within ±0.2° due to differences in equipments used for measurement, measurement environments, and data analysis methods. In addition, the relative intensity of each peak in the X-ray powder diffraction pattern can vary depending on the crystal habit, sampling conditions, and measurement conditions.

The identity of crystal by the X-ray powder diffraction is determined by comparing diffraction angles and overall diffraction patterns between the crystalline form according to one embodiment of the present invention and a subject crystalline form. When they are same as or very similar with each other, for example, when their diffraction angles have angle identities of 7 or more, preferably of 8 or more, more preferably of 9 or more, and even more preferably of 10 all in the above diffraction angles, it can be said that the subject crystalline form has the same identity as the crystalline form according to one embodiment of the present invention.

Another aspect of the present invention is a crystalline form of anserine characterized by a melting point of 240±2° C. by differential thermal analysis.

The value of melting point (extrapolated onset or extrapolated melting onset temperature) by differential thermal analysis (TG-DTA) may have an error within ±2° C. due to differences in the equipments used for measurement, measurement environments, and sample volumes.

The identity of crystal by TG-DTA is determined by comparing the melting points and DTA curve patterns between the crystalline form according to one embodiment of the present invention and a subject crystalline form. When they are same as or very similar with each other, for example, when the melting points are 240±2° C. and the patterns are generally close, it can be said that the subject crystalline form has the same identity as the crystalline form according to one embodiment of the present invention.

Another aspect of the present invention is a crystalline form of anserine characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X- ray (CuKα radiation) as well as a melting point of 240±2° C. by differential thermal analysis.

The X-ray powder diffraction measurement and differential thermal analysis for the crystalline form of anserine are performed by the methods described in Examples below.

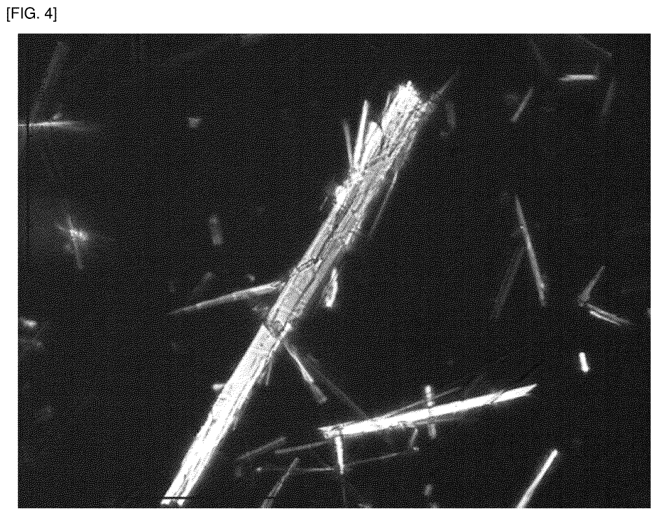

Other properties of the crystalline form according to one embodiment of the present invention are not limited as long as the diffraction angles of diffraction peaks in the X-ray powder diffraction pattern and/or the melting point by differential thermal analysis is within the above-mentioned range. The crystalline form according to one embodiment of the present invention tends to exhibit the shape shown in FIG. 4 when subjected to microscopic observation with a polarizer. Therefore, in addition to comparisons of the diffraction angles of diffraction peaks in the X-ray powder diffraction pattern and/or the melting points by differential thermal analysis, when by microscopic observation using a polarizer, their shapes are same as or very similar with each other, it may be determined that the subject crystalline form has the same identity as the crystalline form according to one embodiment of the present invention.

The purity of anserine in the crystalline form according to one embodiment of the present invention is not particularly limited as long as the diffraction angles of diffraction peaks in the X-ray powder diffraction pattern and/or the melting point by differential thermal analysis is within the above-mentioned range. Examples of the purity include preferably 99.8% or more, and more preferably 99.9% or more. The upper limit of the purity of anserine in the crystalline form according to one embodiment of the present invention is typically 100%. The purity of anserine is preferably within the range between 99.8% and 100%, and more preferably in the range between 99.9% and 100%.

The crystalline form according to one embodiment of the present invention has preferably less purity of contaminants. Major contaminants include creatinine and carnosine, which are difficult to be separated from anserine. The purity of creatinine in the crystalline form according to one embodiment of the present invention is preferably less than 0.1%, more preferably less than 0.05%, and even more preferably substantially 0.0%. The purity of carnosine in the crystalline form according to one embodiment of the present invention is preferably less than 0.2%, more preferably 0.14% or less, and even more preferably 0.13% or less. The crystalline form according to one embodiment of the present invention preferably has a purity between 0% or more and less than 0.1% for creatinine and a purity between 0% or more and less than 0.2% for carnosine; more preferably has a purity between 0% or more and less than 0.05% for creatinine and a purity between 0% and 0.14% for carnosine; and even more preferably has a purity of substantially 0.0% for creatinine and a purity between 0% and 0.13% for carnosine.

In view of the purities of anserine, creatinine and carnosine, the crystalline form according to one embodiment of the present invention has preferably a purity of 99.8% or more for anserine, a purity less than 0.05% for creatinine, and a purity less than 0.2% for carnosine (provided that the total purity of creatinine and carnosine is less than 0.2%). The crystalline form according to one embodiment of the present invention has more preferably a purity of 99.9% to 100% for anserine, a purity between 0% or more and less than 0.05% for creatinine, and a purity of 0% to 0.13% for carnosine (provided that the total purity of creatinine and carnosine is less than 0.2%).

### Method According to One Embodiment of the Present Invention

One embodiment of the present invention is a method of producing a crystalline form of anserine. According to the method according to one embodiment of the present invention, the crystalline form according to one embodiment of the invention can be produced.

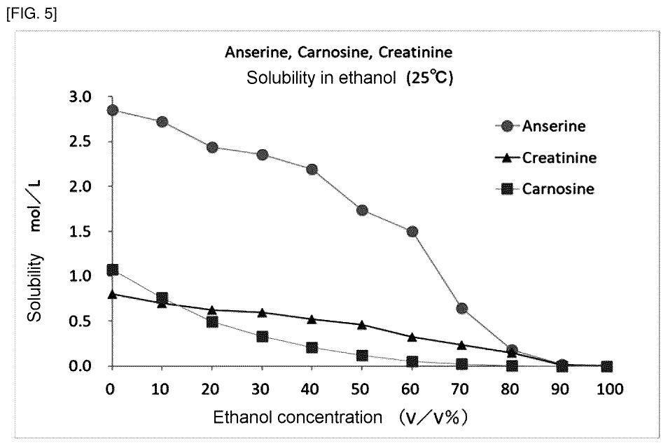

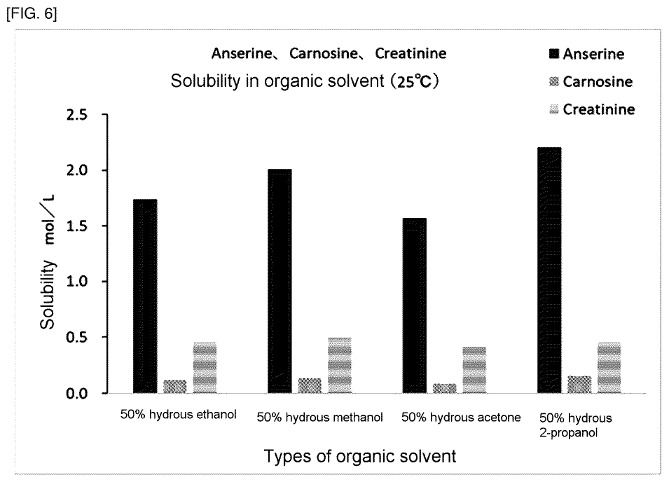

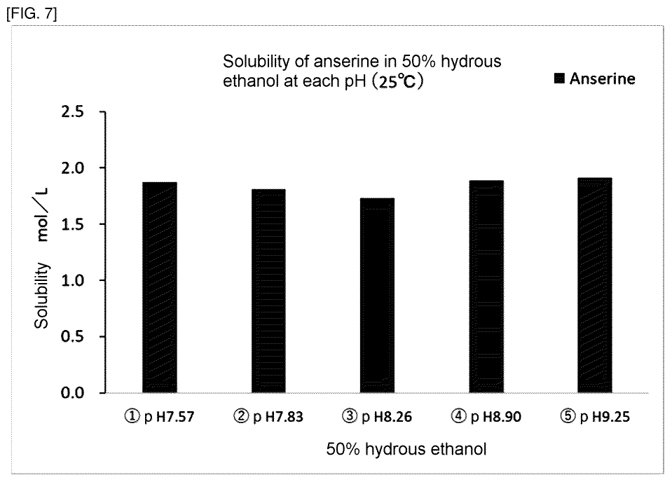

As shown in FIGS. 5 to 6, anserine has high solubility in water and hydrous organic solvent containing a lower concentration of organic solvent. As shown in FIG. 7, the solubility of anserine decreases at the lowest level around the isoelectric point pH 8.3 while the solubility can be hardly affected by pH changes in the range of pH 7.57 to pH 9.25 and thus is nearly constant regardless of pH values. Therefore, the methods described in Patent Documents 2 and 3 that are a method of obtaining a crystalline form of carnosine, which is one of imidazole dipeptides like anserine, cannot produce a crystalline form of anserine.

The method according to one embodiment of the present invention is invented by focusing on such solubility of anserine. The method according to one embodiment of the present invention is a method of producing a crystalline form of anserine by instantaneously adding to an aqueous solution containing a crude anserine organic solvent at such an amount that the final concentration becomes a predetermined high concentration to obtain a slurry, removing liquid components from the slurry to obtain a cake, and then washing the cake using hydrous organic solvent containing a high concentration of organic solvent to obtain a crystalline form of anserine.

The method according to one embodiment of the present invention includes the steps of: instantaneously adding to an aqueous solution containing a crude anserine with a purity of 70% or more (hereinafter also referred to as “aqueous crude anserine solution”) organic solvent at such an amount that the final concentration becomes 70% or more to obtain a slurry (hereinafter also referred to as “slurry step”); subjecting the slurry to solid-liquid separation treatment to obtain a cake (hereinafter also referred to as “cake step”); and subjecting the cake to washing treatment using 85% or more hydrous organic solvent to obtain a crystalline form of anserine (hereinafter also referred to as “washing step”).

The aqueous crude anserine solution is, depending on its properties, preferably subjected to pretreatment in the slurry step in order to efficiently produce a crystalline form of anserine. As such, the method according to one embodiment of the present invention preferably includes a step of subjecting the aqueous crude anserine solution to pretreatment prior to the slurry step (hereinafter also referred to as “pretreatment step”).

In the following, the method according to one embodiment of the present invention will be explained separately for the pretreatment step, the slurry step, the cake step, and the washing step.

### [Pretreatment Step]

The method according to one embodiment of the present invention employs the aqueous crude anserine solution as a starting material. The purity of crude anserine in the aqueous crude anserine solution is not particularly limited as long as the purity is equal to or more than 70%. Examples of the purity include preferably 75% or more, and more preferably 80% or more from the viewpoint of improving the purity of the crystalline form of anserine finally obtained. The upper limit of the purity of crude anserine is not particularly limited, but typically 99%. The purity of crude anserine is preferably between 75% and 99%, and more preferably between 80% and 99%.

Since the solubility of anserine in water is relatively higher, the aqueous crude anserine solution can be obtained by adding a crude anserine to water and then mixing the mixture. The anserine content in the aqueous crude anserine solution is not particularly limited. Examples of the content includes preferably 5% by mass to 20% by mass, and more preferably about 10% by mass from the viewpoint of improved dispersibility of anserine in the aqueous solution.

The method of obtaining the aqueous crude anserine solution is not particularly limited and may be any methods, for example, a method including dissolving a solid crude anserine in water, and a method including obtaining the aqueous crude anserine solution from an anserine-containing material. As such a solid crude anserine, the commercially available crude anserine such as “L-anserine nitrate” (Sigma-Aldrich, 98% purity) may be used. Examples of the method of obtaining the aqueous crude anserine solution from an anserine-containing material include the method described in Example 5 of Patent Document 1 disclosed by the present inventors.

The method described in Example 5 of Patent Document 1 is a method of obtaining an anserine-containing liquid product from an extract of animals such as chicken and salmon (animal extract). When using this method, the aqueous crude anserine solution can be obtained with a content of about 10% by mass and with a purity of about 80%.

The animal extract may be obtained by dissolving components contained in meats and other parts of fishes, birds, mammals and other animals in an extracting medium. The type of animal is not particularly limited as long as it is an animal that contains anserine in its meats or other parts. Examples of the animal include bonito, tuna, salmon, eel, shark, cattle and chicken that are abundant in anserine. The animal extract is preferably derived from meats from livestock animals such as chicken and cattle, and muscles from fishes such as salmon, bonito and tuna, since the animals are abundant in anserine, and are largely present in terms of resources or are easy to breed.

The method of obtaining the animal extract is not particularly limited. Any extracts obtained by subjecting the animal parts containing anserine to known extraction methods such as water extraction, hot water extraction and supercritical extraction, or those commercially available may be used. The animal extract is preferably subjected to any processing treatment such as solid-liquid separation treatment, concentration treatment, drying treatment and dilution treatment in order to remove any insoluble solids and contaminants from the above extract.

The following is a summary of the method of obtaining an anserine-containing liquid product from meats of salmon, chicken or other animal according to the method described in Example 5 of Patent Document 1.

Parts of salmon, chicken or other animal added to water are subjected to hot water extraction treatment at a temperature in the range between 80° C. and 95° C. for tens of minutes to several hours. The resulting hot water extract is subjected to solid-liquid separation treatment and optionally to desalting treatment using electrodialysis or a nanofiltration membrane to obtain an animal extract with 0.1% by mass to 1.0% by mass of anserine, 1.0% to 10.0% of Brix, and pH 5.6 to pH 8.0.

The animal extract is passed at 1 RV to 10 RV and SV 1 to SV 3 through a column packed with a strongly acidic cation exchange resin converted to Na type, and water is then passed through the column at 0.5 RV to 5 RV to adsorb anserine in the animal extract onto the strongly acidic cation exchange resin. The pH value in the column after this adsorption treatment is 5.6 to 8.2.

Then, 0.1 N to 1.0 N alkali metal salt hydroxide solution is passed through the column at 1 RV to 5 RV and SV 1 to SV 5 to obtain a highly pure imidazole dipeptide as an eluate (animal extract treatment solution). The pH value in the column after this elution treatment is 8.5 to 15.0.

The resulting animal extract treatment solution is adjusted to pH 8 to pH 9 by adding an acid thereto, and then passed through a column packed with an aromatic hydrophobic adsorption resin under the conditions of 10° C. to 30° C., 1 RV to 10 RV and SV 1 to SV 5 to adsorb anserine in the animal extract treatment solution onto the aromatic hydrophobic adsorption resin. The pH value in the column after this adsorption treatment is 8 to 9, as with the animal extract treatment solution used.

Then, 0.001 M to 0.01 M aqueous alkali metal salt hydroxide solution as a dilute alkaline solution is passed through the column under the conditions of 10° C. to 30° C., 1 RV to 10 RV and SV 1 to SV 5, so that several types of imidazole dipeptides are separated mutually from each other, and anserine-rich fractions with every appropriate amount are collected to obtain an anserine-containing liquid product containing a high purity of anserine. The pH value in the column after the elution treatment is 8 to 12, as with the dilute alkaline solution used. The anserine-containing liquid product may be subjected to additional treatment such as pH adjustment treatment, desalting treatment, concentration treatment and sterile filtration treatment.

If the anserine-containing liquid product obtained from the animal extract as described above is adjusted with hydrochloric acid such that the pH value becomes neutral, a portion of anserine exists as the hydrochloride salt. As a result, even when directly crystallizing the product, a free crystalline form of anserine cannot be obtained. Therefore, in order to obtain the free crystalline form of anserine, chloride ions in the aqueous crude anserine solution are removed. Similarly, in order to obtain the free crystalline form of anserine from the commercially available anserine nitrate crystal, nitrate ions are removed.

The method of removing chloride ions from the aqueous crude anserine solution is not particularly limited but is preferably a method including removing the majority of chloride ions from the aqueous crude anserine solution. However, according to findings by the present inventors, it was assumed that anserine and chloride ions in an anserine hydrochloride solution were electrically bonded to each other, so that it was necessary to separate them using a stronger electrical force. After repeated trial and error, the present inventors found out that the majority of chloride ions could be removed from the aqueous crude anserine solution by utilizing ion exchange treatment using an appropriate amount of OH type strongly basic anion exchange resin for a neutralized anserine solution. From this perspective, the aqueous crude anserine solution containing a large amount of chloride ions is preferably subjected to ion exchange treatment using an OH type strongly basic anion exchange resin.

The anion exchange resin is an ion exchange resin having an anionic ion exchange group. The anion exchange resins are broadly divided into two types: strongly basic anion exchange resins having strongly basic ion exchange groups such as quaternary ammonium group including trimethylammonium group and dimethylethanolammonium group; and weakly basic anion exchange resins having weakly basic ion exchange groups such as primary and secondary amino groups. Among them, the ion exchange treatment of the aqueous crude anserine solution preferably uses the strongly basic anion exchange resin, and preferably uses the strongly basic anion exchange resin having trimethylammonium group or dimethylethanolammonium group as the ion exchange group.

The strongly basic anion exchange resin may be produced by known methods or commercially available. Commercially available strongly basic anion exchange resins include those sold under the brand names “DIAION” (Mitsubishi Chemical); “Amberlite” (Organo); “Dowex”, “Muromac” and “Levacit” (Muromachi Chemical). Specific examples thereof includes “DIAION SA10A”, “DIAION SA20A”, “Amberlite IRA402BL”, “Amberlite IRA410J”, “Dowex Marathon A”, “Dowex Marathon A2”, “MUROMAC XSA-2613”, “MUROMUC XSB-2613,” “Levacit Monoplus M500,” and “Levacit Monoplus M600”.

Before the strongly basic anion exchange resin is bought into contact with the aqueous crude anserine solution, the ion exchange group of the strongly basic anion exchange resin is converted to the OH type. When the ion exchange group is already in the form of OH type, it can be used as is. When the ion exchange group is in the other form than OH type, it is converted to the form of OH type. The method for converting the ion exchange group to the OH type is not particularly limited. Examples of the method include a method including converting a strongly basic anion exchange resin to the OH type by immersing or passing the resin through a solution containing a hydroxide. The hydroxide is not particularly limited. Examples of the hydroxide include sodium hydroxide and potassium hydroxide. The hydroxide is preferably sodium hydroxide in view of general use and economic efficiency.

For example, the conversion of the strongly basic anion exchange resin to the OH type can be achieved by passing 0.5 N to 2 N aqueous sodium hydroxide solution at 1.5 RV to 3.0 RV through the column packed with the strongly basic anion exchange resin, and then passing a sufficient amount of water through the column.

The method for bringing the aqueous crude anserine solution into contact with the strongly basic anion exchange resin is not particularly limited as long as their contact allows chloride ions in the aqueous crude anserine solution to be adsorbed onto the strongly basic anion exchange resin. The method may be any methods including a batch method including immersing the strongly basic anion exchange resin in the aqueous crude anserine solution, or a column method including passing the aqueous crude anserine solution through the column packed with the strongly basic anion exchange resin. For example, when the aqueous crude anserine solution is passed through the column packed with the strongly basic anion exchange resin, the aqueous crude anserine solution may be passed at a temperature of 10° C. to 40° C., preferably at room temperature between 15° C. and 25° C., such that SV becomes in the range between 0.5 and 8, preferably between 1 and 3.

The solution collected after bringing the aqueous crude anserine solution into contact with the strongly basic anion exchange resin is used as the ion exchange treatment solution. The content of chloride ions in the ion exchange treatment solution is not particularly limited as long as the content is less than that in the aqueous crude anserine solution before subjected to the ion exchange treatment. Examples of the content is preferably 5% or less, more preferably 0% to 3%, and even more preferably 0% to 1% relative to solid content (dry mass) of the ion exchange treatment solution. By removing chloride ions, the purity of anserine in the ion exchange treatment solution is relatively higher than that in the aqueous crude anserine solution.

The aqueous crude anserine solution and the ion exchange treatment solution contain a large amount of water. Therefore, in order to form a slurry by adding organic solvent to them, a large amount of organic solvent may be added. Thus, the aqueous crude anserine solution and the ion exchange treatment solution are preferably subjected to concentration treatment to reduce the amount of organic solvent added.

The concentration treatment is not particularly limited as long as the treatment applies volatilizing or removing water from the aqueous crude anserine solution and the ion exchange treatment solution. The concentration treatment may take any known concentration treatment directed to a solution. The concentration treatment is preferably decompression concentration treatment at room temperature or in a heated state using an evaporator from the viewpoint that anserine is dissolved with good dispersibility in the aqueous crude anserine solution and the ion exchange treatment solution, and furthermore the above treatment is common and simple.

The extent of concentration by the concentration treatment is not particularly limited. Examples of the extent include the extent that the volume becomes reduced to the range between 10% and 99% as compared to that before concentration. The concentration treatment is carried out preferably until Brix becomes within the range between 40% and 80%, and more preferably until Brix becomes within the range between 50% and 70% in view of efficiently forming a slurry.

The concentration solution obtained by the concentration treatment exhibits a liquid form having a candy-like viscosity. However, no anserine crystal is observed.

### [Slurry Step]

A powdered crystalline form of anserine can be obtained by instantaneously adding to the aqueous crude anserine solution or ion-exchange treatment solution, or the concentration solution thereof (they are also collectively referred to as “concentration solution and the like”) organic solvent at such an amount that the final concentration reaches a predetermined high concentration. The progression of crystallization further causes a slurry in which the crystalline form of anserine is suspended in the solution to become the sludgy state.

The organic solvent is not particularly limited as long as it hardly or never dissolves anserine but has a property of being miscible with water. Examples of the organic solvent include alcohol solvent such as methanol, ethanol, 2-propanol; and ketone solvent such as acetone. The organic solvent is preferably ethanol from the viewpoint of lower boiling point, which results in separation from the crystallized anserine, as well as high safety. The organic solvent may be either any one or a combination of two or more of such organic solvent.

The amount of the organic solvent added is such an amount that the concentration of the organic solvent in the mixture obtained after added to the concentrated solution and the like (final concentration; volume ratio) becomes 70% or more. For example, if the amount of the concentration solution and the like is 30 mL, the amount of the organic solvent added is 70 mL or more. As such, the amount of the organic solvent added is not particularly limited as long as the final concentration of the organic solvent becomes 70% or more. Examples of the amount include such an amount that the final concentration becomes 70% to 99%, and from the viewpoint of efficient crystallization of anserine, preferably such an amount that the final concentration becomes 70% to 90%, and more preferably such an amount that the final concentration becomes about 80%. The organic solvent may also be hydrous organic solvent containing water as long as the amount of the organic solvent added is within the above-mentioned range.

The organic solvent is instantaneously added to the concentration solution and the like in order that the organic solvent quickly deprives hydration water surrounding anserine in the concentration solution and the like to expose and crystallize anserine. The slurry cannot be obtained by simply gradually adding the organic solvent. It is a general common technical knowledge in the art that the morphology of crystal formation varies depending on the addition manner of organic solvent. For this purpose, it is preferable to add the entire amount of the prepared organic solvent to the concentration solution and the like at one time. If it is difficult to add the entire amount of the organic solvent to the concentration solution and the like at one time, it may be added in two or three times. In this case, the entire amount is added preferably within a few minutes, more preferably within tens of seconds, and even more preferably within a few seconds.

During and/or after mixing the organic solvent into the concentration solution and the like, the resulting mixture is preferably stirred, and more preferably stirred vigorously. The stirring may be carried out to the extent that the crystalline form of anserine can be observed, either manually with a glass rod or mechanically with a stirring device. The stirring is preferably carried out to the extent that a white clump (the crystalline form of anserine) can be observed in the mixture. The temperature during adding the organic solvent and stirring the solution is not particularly limited but may be room temperature.

It can be recognized that in the solution in which the crystalline form of anserine can be observed, the supersaturation of anserine is gradually resolved to grow a crystal, even when the solution is left to stand. Therefore, the mixture with the white clump generated by adding the organic solvent and stirring the solution, and preferably vigorously stirring the solution, is preferably left to stand. The standing time is not particularly limited. Examples of the standing time include several minutes to several tens of hours, preferably 30 minutes to 10 hours, and more preferably 3 hours to 8 hours.

When stirring, preferably vigorously stirring, the solution again after standing, the crystalline form of anserine agglomerates and then froms an overall whitish, sludgy and thin yogurt-like slurry. The content of anserine in the slurry is not particularly limited. Examples of the content include preferably 5% by mass or more, and more preferably 5% by mass to 20% by mass.

### [Cake Step]

The slurry is subjected to solid-liquid separation treatment to remove liquid components to obtain a cake which is a wet solid.

The solid-liquid separation treatment may be carried out according to solid-liquid separation methods known so far. Examples of the solid-liquid separation method include, but are not limited to, filtration, centrifugation, and pressing. Specifically, the slurry may be subjected to filtration treatment using a membrane filter at room temperature to obtain the cake.

### [Cleaning Step]

When the aqueous crude anserine solution obtained from the animal extract is used, creatinine is contained in the crystalline form of anserine obtained through the slurry step and the cake step. For this reason, the purity of anserine in the crystalline form may not reach 99% or more. In this case, the cake is subjected to washing treatment to remove creatinine and other contaminants. The washing treatment is preferably carried out at least twice depending on the purpose, including first washing treatment and second washing treatment. In other words, the washing treatment is preferably carried out by the first washing treatment and the second washing treatment in a sequential manner.

The first washing treatment employs organic solvent which can dissolve creatinine. However, since the solubility of creatinine decreases in a high concentration of organic solvent, hydrous organic solvent is used. If the concentration of organic solvent is low, anserine may also be dissolved. Therefore, the first washing treatment employs the hydrous organic solvent in which the concentration of organic solvent is 85% or more, preferably 85% to 94%, and more preferably about 90%.

On the other hand, the cake subjected to the first washing treatment may have the remaining creatinine to be adhered onto the cake surface. Therefore, the cake subjected to the first washing treatment is preferably subjected to the second washing treatment using hydrous organic solvent with a concentration at which the solubility of anserine is low. The second washing treatment employs preferably hydrous organic solvent with a higher concentration of organic solvent than that employed in the first washing treatment, preferably 95% to 99.9% hydrous organic solvent, and more preferably 98% to 99% hydrous organic solvent. The organic solvent itself may be used in place of the hydrous organic solvent. It is preferable that the first washing treatment uses 85% to 94% hydrous organic solvent while the second washing treatment uses 95% to 99.9% hydrous organic solvent.

The solvent used in the washing treatment is not particularly limited as long as it is solvent which can dissolve creatinine. Examples of the solvent include the organic solvent listed in the section of the slurry step. From the viewpoint of dissolving creatinine which has been dissolved in water, the solvent is preferably organic solvent miscible with water, preferably methanol, ethanol, 2-propanol and acetone, and in view of general use and safe handling, more preferably ethanol. The organic solvent may be used either individually or in combination of two or more of such organic solvent. In addition, the solvent used in the washing treatment may be the same as or different from the solvent used in the slurry step. Similarly, the solvent used in the first washing treatment may be the same as or different from the solvent used in the second washing treatment. From the viewpoint of safety, each solvent used in the first and second washing treatments is preferably hydrous ethanol. Therefore, the washing treatment is preferably washing treatment to be carried out by washing with 85% to 94% hydrous ethanol followed by washing with 95% to 99.9% hydrous ethanol in a sequential manner.

The washing treatment is not particularly limited as long as it is performed so as to bring the cake in contact with the hydrous organic solvent. For example, when the filtration treatment using a membrane filter is employed in the cake step, the first washing treatment can be carried out at room temperature by adding and filtrating 50 mL to 200 mL of 85% to 94% hydrous organic solvent per 10 g of cake to the remaining cake on the membrane filter while the second washing treatment can be carried out at room temperature by adding and filtering the same volume of 95% to 99.9% hydrous organic solvent to the cake.

As such, the crystalline form of anserine can be obtain by subjecting the cake obtained in the cake step to the washing treatment. The crystalline form of anserine after the washing treatment is subjected to drying treatment, pulverization treatment and other treatments to obtain a solid-state crystalline form of anserine. The purity of anserine in the anserine crystal is preferably 99.8% or more, and more preferably 99.9% or more.

The crystalline form of anserine obtained by the method according to one embodiment of the present invention preferably exhibits peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation) and has a melting point of 240±2° C. by differential thermal analysis, and more preferably has a purity of anserine in the range between 99.8% and 100%, and a purity of carnosine in the range between 0% or more and less than 0.2% in addition to the above properties.

The method according to one embodiment of the present invention may include various steps and operations before, after, or during the above steps as long as it can solve the problems of the present invention.

### Specific Embodiment of the Method According to the Present Invention

While a specific embodiment of the method of producing a crystalline form of anserine will be described below, the method according to the present invention is not limited to the specific embodiment.

The anserine-containing liquid product is obtained by using parts of animal such as salmon and chicken as the raw material according to the method described in Example 5 of Patent Document 1. The anserine-containing liquid product is then passed through a column packed with a strongly basic anion exchange resin converted to OH type at 0.5 RV to 5 RV and SV 1 to SV 3 at room temperature to obtain an ion exchange treatment solution as the passed solution.

The ion exchange treatment solution is then subjected to decompression concentration treatment at a temperature of 40° C. to 60° C. until Brix reaches 40% to 80%. Ethanol is added instantaneously to the resulting concentration solution at room temperature such that the volume ratio of ethanol:concentration solution becomes 99:1 to 70:30. The resulting mixture is vigorously stirred manually or mechanically until a white clump is formed. After stirring, the mixture is left to stand at room temperature for 3 hours to 8 hours followed by stirring vigorously again to obtain a slurry.

The resulting slurry is subjected to filtration treatment using a membrane filter at room temperature to obtain a cake. The cake is then washed by adding and filtrating 50 mL to 200 ml of 85% to 94% hydrous ethanol per 10 g of cake to the cake on the filter at room temperature, and then by adding and filtrating the same volume of 95% to 99.9% hydrous ethanol to the cake at room temperature.

The washed cake is subjected to atmospheric pressure drying treatment at a temperature of 60° C. to 80° C. using an atmospheric pressure dryer followed by pulverization treatment and reduced-pressure drying treatment at a temperature of 50° C. to 70° C. to obtain a crystalline form of anserine in which the purity of anserine is 99.8% or more, e.g., 99.9%.

### Other Aspects of the Present Invention

Another aspect of the present invention is a food composition containing the crystalline form according to one embodiment of the present invention. Another aspect of the present invention is a pharmaceutical composition containing the crystalline form according to one embodiment of the present invention. Another aspect of the present invention is a cosmetic composition containing the crystalline form according to one embodiment of the present invention. The food composition, pharmaceutical composition and cosmetic composition according to one embodiment of the present invention are collectively referred to as “composition according to one embodiment of the present invention.”

The composition according to one embodiment of the present invention may be applied orally or parenterally, depending on its mode of application. Parenteral applications include, but are not limited to, injections and infusions by intradermal, subcutaneous, intravenous and intramuscular administrations; transdermal; and inhalation through mucous membranes such as nose and throat.

The biological individual to which the composition according to one embodiment of the present invention is applied is not particularly limited. Examples of the biological individual include animals including mammals, such as human, dog, cat, cattle, horse, pig and sheep. Among them, the biological individual is preferably human. While the subject individual may be a healthy individual, they are preferably an individual that asks for the recovery from fatigue, the reduction in blood pressure and the other effective actions of anserine.

The composition according to one embodiment of the present invention may include a variety of other components, depending on its mode. Examples of the other component include sugars, flavor enhancers, stabilizers, emulsifiers, starch, processed starch, starch degradation products, seasonings, flavoring agents, colorings, acidifiers, flavor ingredients, nutrients, animal and vegetable ingredients such as fruit juices and eggs, excipients, bulking agents, binding agents, thickening agents, flavorings, preservatives, buffers, lubricants, oil ingredients, moisturizers, coolants, chelating agents, pH adjusters, antioxidants, UV absorbers, UV scatterers, whitening agents, emulsifiers, vitamins, medicinal ingredients, and other additives used in manufacturing foods, pharmaceuticals and cosmetics. The amount of other component used is not particularly limited and can be set appropriately as long as the amount does not interfere to solve the problem of the present invention.

The form of the composition according to one embodiment of the present invention is not particularly limited as long as the form is a commonly used form. Examples of the form include solid, liquid, gel, suspension, cream, sheet, stick, powder, granular, granular, tablet, rod, plate, block, paste, capsule, jelly, and caplet.

While the method of producing the composition according to one embodiment of the present invention is not particularly limited. Examples of the method include a method including mixing the crystalline form of anserine with the other components, and forming the resulting mixture into the desired form.

The composition according to one embodiment of the present invention can be used, for example, as a product containing the high purity of anserine, in applications expecting physiological effects of anserine such as anti-fatigue, antioxidant, hypotensive, anti-inflammatory, and uric acid level lowering effects.

The content of the crystalline form of anserine in the composition according to one embodiment of the present invention is not particularly limited. Examples of the content include preferably 0.001% by mass or more as the dry mass of the crystalline form of anserine relative to the total amount of the composition, and more preferably 0.1% by mass to 99% by mass.

Specific examples of the food composition according to one embodiment of the present invention include, but not limited to, the followings: drinks, such as soft drinks, carbonated drinks, fruit drinks, vegetable juices, lactic acid bacteria drinks, milk drinks, soy milk, mineral water, tea drinks, coffee drinks, sports drinks, alcoholic drinks and jelly drinks; vegetable processed products such as tomato puree, canned mushrooms, dried vegetables and pickles; fruits processed products such as dried fruits, jams, fruit purees and canned fruits; spices such as curry powder, horseradish, ginger, spice blends and seasoning powders; noodles (including fresh and dried noodles) such as pasta, udon, soba noodles, ramen noodles, and macaroni; breads such as breads, sweet breads, prepared breads and doughnuts; flour products such as alphalized rice, oatmeal, fu and batter flour; confectionery such as baked cakes, cookies, rice cakes, candies, chocolates, chewing gums, snack confectionery, chilled desserts, candied confectionery, Japanese cakes, western cakes, semi-baked cakes, pudding and ice cream; bean products such as azuki beans, tofu, natto, soybean flour, yuba (bean curd lees), cooked beans and peanuts; processed foods such as honey and royal jelly; meat products such as ham, sausage and bacon; dairy products such as yogurt, pudding, condensed milk, cheese, fermented milk, butter and ice cream; egg processed products; fish processed foods such as dried fish, kamaboko, chikuwa and fish sausage; processed seaweed such as dried seaweed, kelp and tsukudani; fish egg processed products such as cod roe, herring roe, salmon roe and karasumi; seasonings such as dashi broth, soy sauce, vinegar, mirin, consomme base, Chinese base, concentrated dashi, dressing, mayonnaise, ketchup and miso; edible fats and oils such as salad oil, sesame oil, linoleum oil and diacylglycerol and benibana oil; prepared foods such as soups (including powders and liquids), cooked food, retort food, chilled food and semi-cooked food (e.g., cooked rice stock, crabmeat omelet stock).

The present invention will now be described in further detail with reference to Examples, which are not intended to limit the present invention. The present invention may take various embodiments to the extent that the objectives of the present invention are achieved.

### EXAMPLES

### [Measurement Method]

### (1) Anserine, Creatinine and Carnosine

The anserine content was determined by HPLC. HPLC was performed using the column “InertSustain C18 (particle size 5 μm, φ4.6 mm×150 mm)” (manufactured by GL Sciences), the developing solvent water solution containing 10 mM sodium phosphate (pH 6.5), and the HPLC device “PU-2089” (manufactured by JASCO; flow rate 1.0 mL/min, 25° C., injection volume 5 μL, detection wavelength 210 nm). The measurement was determined according to calibration curve method. The substance “L-anserine nitrate (manufactured by Sigma-Aldrich, 99% purity)” was used as a standard product for quantification. The molecular weight of L-anserine nitrate is 303.27. When L-anserine nitrate is subjected to HPLC, the peaks of L-anserine and nitrate ion peak are separated from each another. The peak area of L-anserin was then used in the calibration curve method. Each quantitative value of anserine was calculated using the molecular weight of L-anserin of 240.26. Similarly, creatinine and carnosine were quantified using “creatinine” (manufactured by FUJIFILM Wako Pure Chemical, 99% purity) and “L-carnosine” (manufactured by Sigma-Aldrich, 99% purity) as standard products, respectively.

### (2) Solid Content and Moisture Content

The solid content and moisture content were determined and calculated by subjecting the solution or solid to decompression drying treatment at 70° C.

### (3) pH, Chloride Ions

The pH value was measured using the pH meter “PH METER D-51” (manufactured by HORIBA). The pH value of the crystalline form of anserine was determined by measuring the 1.0% (w/w) aqueous solution prepared. The level of chloride ions was determined using the device “potentiometric autotitrator” (manufactured by Mitsubishi Chemical Analytec).

### (4) X-Ray Powder Diffraction

The X-ray powder diffraction was performed under the following conditions:
Device: “SmartLab” (manufactured by Rigaku)
Radiation source: CuKα radiation
Temperature: room temperature
Filter: CuK beta filter
X-ray output: 40 kV, 20 mA
Detector: D/teX Ultra
Scanning mode: continuous
Scanning speed: 3°/min
Step width: 0.01° Scanning axis: 2θ/θ
Measuring range (2θ): 10° to 40°
Incident slit: 2/3°
Wavelength: 1.5418 Å
Optics: focusing method
Others: standard sample stage (glass sample plate) and Ni filter used.

### (5) Differential Thermal Analysis (TG-DTA)

The differential thermal analysis was performed under the following conditions:
Device: “TG-DTA2000S” (manufactured by Mac Science)
Temperature raising rate: 10° C./min
Nitrogen flow rate: 200 mL/min
Sample container: aluminum container
Reference: empty sample container

### (6) Microscopic Observation

The microscopic observation was performed using the microscope “MP38T” (manufactured by AS ONE) with the polarizing filters “CIRCULAR P.L. Mark II” (manufactured by Marumi optical) and “polarizing plate” (manufactured by Artec), at a magnification of 400×.

### (7) Total Nitrogen and Total Carbon

The total nitrogen and total carbon were determined conventionally by Dumas method.

### Example 1. Preparation of Anserine-Containing Liquid Product

The 10% imidazole dipeptide-containing liquid product was obtained from white salmon according to the method described in Example 5 of the patent document (JP 6765046 B).

The obtained 10% imidazole dipeptide-containing liquid product (pH 7.0) had 17.3% of Brix, 13.4% (w/w) of solid content, 10.92% by mass of anserine, 0.024% by mass of carnosine, 0.40% by mass of creatinine, and 0.90% by mass of chloride ions. In addition, the purity of anserine was 81.5% based on the amount of anserine per dry mass of the product. The obtained 10% imidazole dipeptide-containing liquid product was referred to as “anserine-containing liquid product.”

### Example 2. Preparation of Anserine Crystal

### (2-1) Example

The column was filled by 150 mL of the strongly basic anion exchange resin “DIAION SA10A” (manufactured by Mitsubishi Chemical). In order to convert the resin to the OH type, 1M sodium hydroxide solution was passed through the column at 2 RV, and then RO water was passed through the column. Then, 200 g of the anserine-containing liquid product (21.8 g of anserine) obtained in Example 1 was subjected to ion exchange treatment by passing the product through the OH type resin-packed column at room temperature at SV 2.0.

The total amount of the passed solution (non-adsorbed fraction) obtained by passing through the column was collected. This solution was referred to as the ion exchange treatment solution (pH 8.83). Since 325.0 g of the ion exchange treatment solution had 5.34% (w/w) of solid content, it was found that the ion exchange treatment solution contained 17.37 g of solids, 15.21 g of anserine, 0.033 g of carnosine, and 0.56 g of creatinine. Based on the amount of anserine per solids, the purity of anserine was 87.6%. On the other hand, the ion exchange treatment solution had chloride ions below the detection limit.

The resulting ion exchange treatment solution was subjected to decompression concentration treatment using an evaporator at 55° C. until Brix reached 60%.

The resulting concentration solution was then subjected to ethanol treatment by adding ethanol to the solution instantaneously at room temperature such that the volume ratio of ethanol: the concentration solution became 80:20, and then by stirring the mixture vigorously with a glass rod to obtain a uniform pale white solution. The solution was further being stirred to form a white clump (floc) in the solution. The solution was then left to stand at room temperature for 5 hours followed by being stirred vigorously again to form a white precipitate. The ethanol treatment solution having the white precipitate obtained in this way was a pale yogurt-like solution as a whole.

It was assumed that the rapid ethanol dispersion was achieved by instantaneously adding ethanol to the concentration solution. It was also presumed that the concentration solution was dispersed into the ethanol layer as fine droplets so that hydration water surrounding anserine was promptly deprived to precipitate anserine out.

The resulting solution having the white precipitate was filtered through a filter paper to obtain a solid cake on the filter paper. The obtained solid cake was subjected to first cake washing treatment by adding 100 mL of 90% hydrous ethanol to the solid cake. The solid cake was then subjected to second cake washing treatment by adding 100 mL of 99% ethanol to the solid cake on the filter paper. The repeated cake washing treatments were performed in this manner to dissolve and remove creatinine contained in the cake.

The cake washed was dried at 70° C. for 3 hours using an atmospheric pressure dryer. The resulting dried matter was ground using a mortar, and the resulting ground matter was then dried under reduced pressure at 60° C. for 4 hours to obtain a crystalline form of anserine. In the resulting anserine crystal, the purity of anserine was 99.9%, and the purity of carnosine was 0.13%. The resulting anserine crystal was called Anserine crystal I.

The yield and purity of anserine in each step were as listed in Table 1.

TABLE 1

| | Anserine | | | Carnosine | | | Creatinine | | | Chloride ions | | |
|---|---|---|---|---|---|---|---|---|---|---|---|---|
| | | | Recovery | | | Recovery | | | Recovery | | | Recovery |
| | Purity | Yield | rate | Purity | Yield | rate | Purity | Yield | rate | Purity | Yield | rate |
| Sample | [%] | [g] | [%] | [%] | [g] | [%] | [%] | [g] | [%] | [%] | [g] | [%] |
| Anserine-containing liquid | 81.5 | 21.84 | 100 | 0.18 | 0.048 | 100 | 2.99 | 0.80 | 100 | 6.70 | 1.80 | 100 |
| product | | | | | | | | | | | | |
| Ion exchange treatment | 87.6 | 15.21 | 70 | 0.19 | 0.033 | 69 | 3.23 | 0.56 | 70 | 0.00 | 0.00 | 0 |
| solution | | | | | | | | | | | | |
| Ethanol treatment solution: | 60.1 | 2.11 | 10 | 0.57 | 0.020 | 42 | 13.40 | 0.47 | 59 | 0.00 | 0.00 | 0 |
| supernatant | | | | | | | | | | | | |
| Cake solid: | 97.2 | 12.93 | 59 | 0.12 | 0.016 | 33 | 0.96 | 0.13 | 16 | 0.00 | 0.00 | 0 |
| before cake washing treatment | | | | | | | | | | | | |
| Cake solid: | 99.9 | 11.66 | 53 | 0.13 | 0.015 | 31 | 0.00 | 0.00 | 0 | 0.00 | 0.00 | 0 |
| after cake washing treatment | | | | | | | | | | | | |

Anserine crystal II, Anserine crystal III, and Anserine crystal IV were obtained in the same manner as Anserine crystal I.

### (2-2) Comparative Example

When the ion exchange treatment solution was subjected to decompression concentration treatment until Brix reached 80%, the resulting concentrate was highly viscous, and the addition of ethanol caused much less dispersibility in the ethanol layer, resulting in a small paste-like clump but no crystal. It was presumed that as the concentration rate was grater, the hydration power was stronger thereby suppressing dehydration by ethanol.

When the concentration solution was subjected to ethanol treatment, ethanol was added to the concentration solution having 50% of Brix such that the volume ratio of ethanol: the concentration solution reached 20:80, resulting in the significantly decreased amount of crystal formed and the significantly lower recovery rate. It was assumed that this was caused by the significantly high solubility of anserine in water.

According to the method described in Example 5 of the patent document (JP 6765046 B), the solution obtained by subjecting the eluate to pH adjusting treatment (hereinafter referred to as “anserine neutralization eluate”) was subjected to decompression concentration treatment with no ion exchange treatment performed. The resulting concentrate was a candy-like paste and could not be dried and solidified. In addition, when freeze-drying treatment was performed instead of decompression concentration treatment, the resulting concentrate was also a candy-like paste and could not be dried and solidified. It was presumed that this was caused by the significantly high hydration power of anserine.

When the anserine neutralization eluate was subjected to decompression concentration treatment with no ion exchange treatment performed, and then was subjected to ethanol treatment, the resulting precipitate was a gum-like paste to be very difficult to collect. The precipitate collected was concentrated, and dried and solidified, resulting in a hard clump. It was presumed that when chloride ions were present in the concentrate, anserine could not be crystallized but became in the gum-like form.

In the cake washing treatment, when the concentration of ethanol in the hydrous ethanol used was lower, the cake was dissolved, and the yield rate decreased. Furthermore, when the cake washing was insufficient, creatinine was present as a contaminant, and no highly pure crystalline form of anserine could be obtained.

### (2-3) Reference Example

L-carnosine (manufactured by Sigma-Aldrich) was completely dissolved in water, and the resulting aqueous solution was then subjected to decompression concentration treatment, resulting in a precipitation at the time that Brix reached 30%. The decompression concentration treatment was further continued, resulting in a dry solid. The dry solid showed a crystalline form under the microscopic observation. As seen above, it was found that the carnosine crystal could be obtained as a precipitate by simply subjecting the aqueous carnosine solution to decompression concentration treatment. This result was consistent well with the disclosure of Patent Document 2.

In contrast, even when the anserine crystal was completely dissolved in water and then the resulting solution was subjected to decompression concentration treatment, only a candy-like paste was obtained while no precipitate of crystal was formed.

From the above, it was found that anserine could not be crystallized according to the method described in Patent Document 2. It was found that the method of Example 2 above was a method suitable for crystallizing anserine, since carnosine could be crystallized regardless of the method of Example 2.

### Example 3. Evaluation of Physical Properties of Anserine Crystal

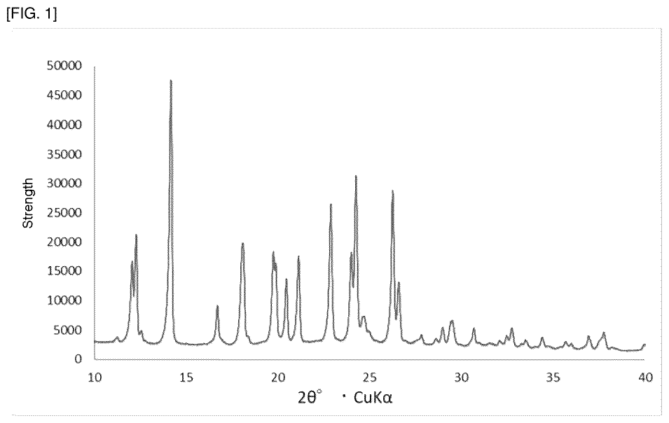

Anserine crystals I to IV were analyzed by X-ray powder diffraction. FIG. 1 shows the X-ray powder diffraction pattern of Anserine crystal III. In addition, Table 2 summarizes characteristic peaks shared among Anserine crystals I to IV. The d-spacings and relative integrated intensities in Table 2 were determined using average values of Anserine crystals I to IV.

TABLE 2

| | | | Relative integrated |
|---|---|---|---|
| | Diffraction angles | D-spacings | intensities |
| Peak No. | (2θ) | (Å) | (>10%) |
| 1 | 12.0 | 7.3 | 25 |
| 2 | 12.3 | 7.2 | 42 |
| 3 | 14.1 | 6.3 | 100 |
| 4 | 18.1 | 4.9 | 35 |
| 5 | 21.1 | 4.2 | 36 |
| 6 | 22.9 | 3.9 | 55 |
| 7 | 24.0 | 3.7 | 29 |
| 8 | 24.2 | 3.7 | 56 |
| 9 | 26.2 | 3.4 | 54 |
| 10 | 26.5 | 3.4 | 22 |

Anserine crystals I to IV showed characteristic peaks at 12.0°, 12.3°, 14.1°, 18.1°, 21.1°, 22.9°, 24.0°, 24.2°, 26.2° and 26.5° as diffraction angles (2θ) by X-ray powder diffraction, as shown in Table 2.

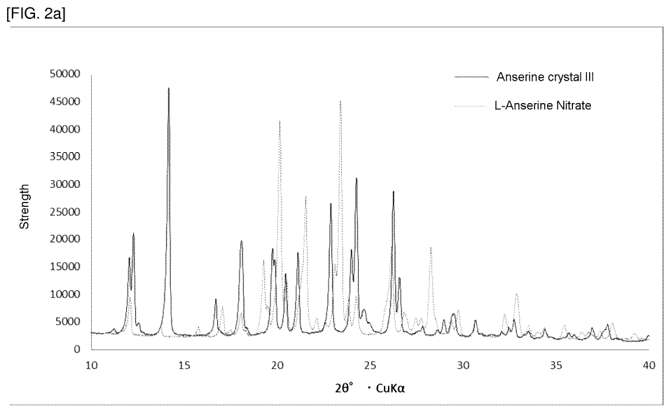

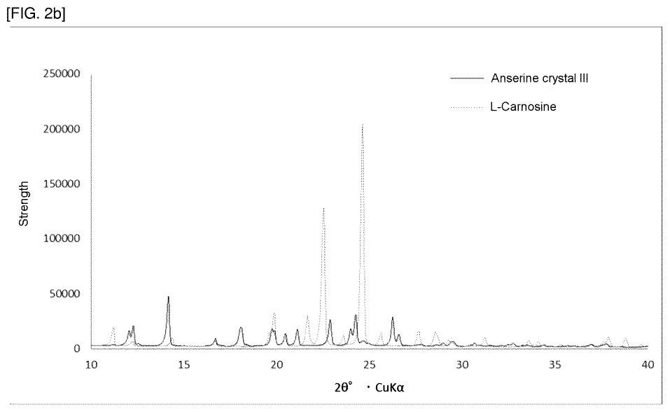

In addition, the commercially available standard products “L-Anserine Nitrate” (manufactured by FUJIFILM Wako Pure Chemical, purity 98.8%) and “L-Carnosine” (manufactured by Sigma-Aldrich) were analyzed by X-ray powder diffraction to obtain each diffraction pattern. The comparisons of the above diffraction patterns with the diffraction pattern of Anserine crystal Ill are shown in FIG. 2A and FIG. 2B, respectively. As shown in these figures, Anserine crystal Ill exhibited a different diffraction pattern from those of the commercially available anserine and carnosine.

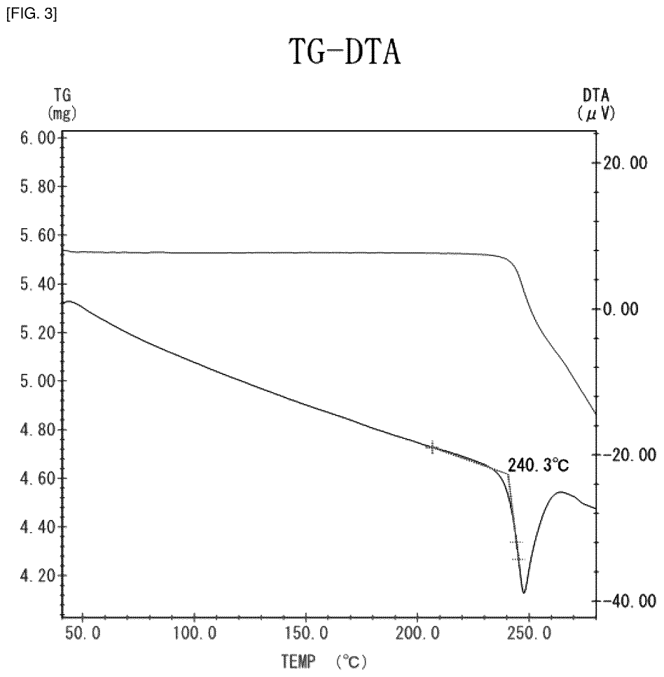

The melting points of the anserine crystals I to IV were determined by differential thermal analysis. The data of differential thermal analysis for Anserine crystal Ill is shown in FIG. 3. The melting points of Anserine crystals I to IV were 240±2° C. as the result of differential thermal analysis.

The result of microscopic observation for Anserine crystal II is shown in FIG. 4. In addition, the results of measuring solid content, moisture content, total nitrogen and total carbon for Anserine crystal II are shown in Table 3.

TABLE 3

| Items | Measurement results |
|---|---|
| Solid content | 99.96% |
| Moisture content | 0.04% |
| Total nitrogen | 23.5% |
| Total carbon | 50.1% |

FIG. 4 shows a polarizing microphotograph. This photograph demonstrated that the anserine crystal obtained in Example 2 was a needle-shaped crystal. It was found from the measurement results in Table 3 that the anserine crystal was a solid substance with little moisture content, and furthermore was in the free form but not in the salt state when taking into consideration the total nitrogen content and the total carbon content.

The pH value of the anserine crystal was 8.30. This was consistent with the isoelectric point of anserine.

### Example 4. Evaluation of Solubilities of Anserine, Carnosine and Creatinine

Anserine crystal II, L-carnosine (manufactured by Sigma-Aldrich) and creatinine (manufactured by FUJIFILM Wako Pure Chemical) were saturated in 0% to 99% hydrous ethanol at 25° C. until they were no longer dissolved, respectively. The solubility of each crystal was evaluated at each ethanol concentration.

In other words, each crystalline form of anserine, carnosine and creatinine was placed in a centrifuge tube containing 2 mL of 0% to 99% ethanol solution prepared in increments of 10%. During stirring the centrifuge tube with a touch mixer, the crystalline form was added and dispersed in the ethanol solution until the precipitate was no longer dissolved. The centrifuge tube was then subjected to sonication treatment in an ultrasonic thermostatic bath at 25° C. for 20 minutes. This sonication treatment was repeated four times, and the presence or absence of precipitate was observed. When the absence of precipitate was observed, the crystalline form was additionally introduced.

After the sonication treatment, the centrifuge tube with the precipitate was subjected to centrifugation treatment (at 25° C. and 1,850 G for 5 min), and the resulting supernatant was filtered with 0.45 μm filter (PTFE). The obtained filtrate was analyzed by HPLC to determine the contents of anserine, carnosine and creatinine dissolved.

FIG. 5 shows the results of determining solubility of each crystalline form of anserine, carnosine and creatinine. As shown in FIG. 5, it was indicated that anserine had greater solubility than that of carnosine or creatinine in 0% to 70% of ethanol concentration. From the results, it was found that anserine could not be easily collected by ethanol precipitation, and that the higher concentration of ethanol was suitable for crystallizing anserine.

### Example 5. Evaluation of Solubilities of Anserine, Carnosine and Creatinine in Organic Solvent

Each crystalline form of anserine, carnosine and creatinine was saturated in 50% hydrous ethanol, 50% hydrous methanol, 50% hydrous acetone or 50% hydrous 2-propanol at 25° C. until the crystalline form was no longer dissolved. In this way, the solubility of the crystalline form in each organic solvent was evaluated.

The solubility was evaluated in the same manner as in Example 4, except that 50% hydrous ethanol, 50% hydrous methanol, 50% hydrous acetone or 50% hydrous 2-propanol was used instead of 0% to 99% hydrous ethanol.

FIG. 6 shows the results of determining solubility of each crystalline form of anserine, carnosine and creatinine. As shown in FIG. 6, the anserine crystal had greater solubility in any organic solvent than that of the carnosine or creatinine crystal. Therefore, it was found that the higher concentration of organic solvent was suitable to collect the precipitate of anserine crystal.

### Example 6. Evaluation of pH Effect on Solubility of Anserine Crystal

The crystalline form of anserine was saturated in 50% hydrous ethanol adjusted at each pH value using hydrochloric acid and sodium hydroxide at 25° C. until the crystalline form was no longer dissolved, and the solubility at each pH value was evaluated.

The solubility of the crystalline form of anserine was evaluated as in Example 4, except for using 50% hydrous ethanol adjusted at a pH value of 7.57, 7.83, 8.26, 8.90 or 9.25. The filtrate obtained with 0.45 μm filter (PTFE) was further prepared to a 1% solution, and the pH value of the solution was measured.

FIG. 7 shows the results of determining solubility of each crystalline form of anserine, carnosine and creatinine. As shown in FIG. 7, the lower solubility of anserine crystal was found at the isoelectric point of anserine (pH 8.3) but there was no significant difference in the solubility in 50% hydrous ethanol with the pH range between 7.6 and 9.3.

### INDUSTRIAL APPLICABILITY

The present invention is useful in the fields of foods and beverages, pharmaceuticals, cosmetics, quasi-pharmaceutical products and the like, and particularly has the advantage of being able to industrially produce a crystalline form of anserine which is available for as an active ingredient an anti-fatigue composition, an antioxidant composition, a blood pressure lowering composition, an anti-inflammatory composition, and an uric acid level lowering composition.

### CROSS-REFERENCING OF RELATED APPLICATIONS

The present application claims the benefit of priority to Japanese Patent Application No. 2021-155549, filed on Sep. 24, 2021, the disclosure of which is incorporated herein by reference in its entirety.

The disclosure of all the documents described herein including Patent Documents 1 to 4 is incorporated herein by reference in its entirety.

1. A crystalline form of anserine, characterized by peaks at diffraction angles (2θ) of 12.0°, 12.3°, 14.1°, 18.1°, 21.1°, 22.9°, 24.0°, 24.2°, 26.2°, and 26.5° in an X-ray powder diffraction pattern obtained using a characteristic X-ray (CuKα radiation).

2. A crystalline form of anserine, characterized by a melting point of 240±2° C. by differential thermal analysis.

3. The crystalline form of anserine according to claim 1, wherein the purity of anserine is equal to or more than 99.8% and/or the purity of carnosine is less than 0.2%.

4. A food composition, pharmaceutical composition or cosmetic composition, comprising the crystalline form according to claim 1.

5. A method of producing a crystalline form of anserine, comprising instantaneously adding to an aqueous solution comprising a crude anserine with a purity equal to or more than 70% organic solvent at such an amount that the final concentration becomes equal to or more than 70% to obtain a slurry;
subjecting the slurry to solid-liquid separation treatment to obtain a cake; and
subjecting the cake to washing treatment using 85% or more hydrous organic solvent to obtain the crystalline form of anserine.

6. The method according to claim 5, wherein the aqueous solution comprising a crude anserine is an aqueous solution comprising a crude anserine obtained from an animal extract.

7. The method according to claim 5, wherein the aqueous solution comprising a crude anserine is an aqueous solution comprising a crude anserine subjected to ion exchange treatment using an OH type strongly basic anion exchange resin.

8. The method according to claim 5, wherein the aqueous solution comprising a crude anserine is an aqueous solution comprising a crude anserine subjected to ion exchange treatment using an OH type strongly basic anion exchange resin and concentration treatment.

9. The method according to claim 8, wherein the concentration treatment is decompression concentration treatment to be carried out until Brix of the aqueous solution becomes in the range from 50% to 70%.

10. The method according to claim 5, wherein the washing treatment is washing treatment in which washing using 85% to 94% hydrous organic solvent and washing using 95% to 99.9% hydrous organic solvent are sequentially carried out.

11. The method according to claim 5, wherein the organic solvent used is each independently at least one organic solvent selected from the group consisting of methanol, ethanol, 2-propanol and acetone.

12. The method according to claim 6, wherein the animal extract is an animal extract derived from at least one animal selected from the group consisting of bonito, tuna, salmon, eel, shark, cattle and chicken.
